(19) United States
(12) Patent Application Publication
(10) Pub. No.: US 2017/0216155 A1
(43) Pub. Date: Aug. 3, 2017

(54) TOOTH BEAN SYSTEMS

(71) Applicant: Rita Maloney, Woodbridge, VA (US)

(72) Inventor: Rita Maloney, Woodbridge, VA (US)

(21) Appl. No.: 15/486,576

(22) Filed: Apr. 13, 2017

Related U.S. Application Data
(63) Continuation-in-part of application No. 14/482,354, filed on Sep. 10, 2014.

(51) Int. Cl. A61K 8/02; A61K 8/97; A61K 8/29; A46B 9/04; A61K 8/60; A61K 8/92; A61K 8/98; A61Q 11/00; A61K 8/34

(52) U.S. Cl. CPC A61K 8/0204; A61Q 11/00; A61K 8/97; A61K 8/29; A61K 8/345; A61K 8/602; A61K 8/922; A61K 8/987; A46B 9/04; A61K 2800/30; A46B 2200/1066

(57) ABSTRACT

A toothpaste system including a toothbrush and a dissolvable bean-shaped toothpaste mixture used to permit a user-consumer to evenly distribute toothpaste all around a mouth portion, teeth, and a gum region of the user-consumer and then brush their teeth with the toothbrush while reducing the amount of toothpaste that gets wasted and thrown away when stored in a tube.

### CROSS-REFERENCE TO RELATED APPLICATION

The present application is a continuation-in-part and is related to and claims priority from prior provisional application Ser. No. 61/711,882, filed Oct. 10, 2012, and pending non-provisional applications Ser. No. 14/049,688 filed Oct. 9, 2013, and Ser. No. 14/482,354 filed on Sep. 10, 2014, which applications are incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of oral hygiene devices and more specifically relates to a dissolvable toothpaste bean-shaped system.

2. Description of the Related Art

Toothpaste is a paste or gel dentifrice used with a toothbrush as an accessory to clean and maintain the aesthetics and health of teeth. Toothpaste is used to promote oral hygiene: it serves as an abrasive that aids in removing the dental plaque and food from the teeth, assists in suppressing halitosis, and delivers active ingredients (most commonly fluoride) to help prevent tooth and gum disease (gingivitis).

Most of the cleaning is achieved by the mechanical action of the toothbrush, and not by the toothpaste. Salt and sodium bicarbonate (baking soda) are among materials that can be substituted for commercial toothpaste. Toothpaste is not intended to be swallowed due to potential fluoride content, but is generally not very harmful if accidentally swallowed in small amounts; however, one should seek medical attention after swallowing abnormally large amounts.

Unfortunately, many people, especially children, make a mess when squeezing toothpaste on their toothbrush. Not only can this be a hassle to clean, but it can also be wasteful. Additionally, tubes of toothpaste are typically only offered in one flavor, which can become boring and repetitive over time.

Various attempts have been made to solve the above-mentioned problems such as those found in U.S. Pat. Nos. and U.S. Publication Nos. 7,354,569; 2005/0260141; 8,113,729; 2008/0038046; 4,238,475; and 6,379,654. This art is representative of portable oral hygiene devices. None of the above inventions and patents, taken either singly or in combination, is seen to describe the invention as claimed.

Ideally, a dissolvable toothpaste bean-shaped system should be easy to use, safe to consume, portable, taste good, look appealing to children, and be manufactured at a modest expense. Thus, a need exists for a dissolvable toothpaste in a bean-shape to permit a user-consumer to evenly distribute toothpaste all around a mouth portion, teeth, and a gum region of the user-consumer while reducing the amount of toothpaste that gets wasted and thrown away when stored in conventional tubes so as to avoid the above-mentioned problems.

### BRIEF SUMMARY OF THE INVENTION

In view of the foregoing disadvantages inherent in the known oral hygiene devices art, the present invention provides a novel dissolvable toothpaste bean-shaped system (entitled Tooth Bean System). The general purpose of the present invention, which will be described subsequently in greater detail, is to provide a dissolvable toothpaste bean-shaped system to permit a user-consumer to evenly distribute toothpaste all around a mouth portion, teeth, and a gum region of the user-consumer while reducing the amount of toothpaste that gets wasted and thrown away when stored in a tube. The instant bean-shaped system is used in conjunction with a toothbrush to achieve improved oral hygiene.

A dissolvable toothpaste bean-shaped system comprising ingredients is disclosed herein combined in proportions substantially including: toothpaste; gum arabic solution go-8-19018; titanium powder, argo 0-200 10-50-0d; pepper mint flavor, triple distilled; xylitol, xylisorb, fine granular; isomalt pf, palatinit; carnauba wax; crystalac c 3.2; and at least one confectioners glaze. The dissolvable toothpaste bean-shaped system is prepared by intermixing the toothpaste; the gum arabic solution go-8-19018; the titanium powder, argo t1-200 10-50-0d; the pepper mint flavor, triple distilled; the xylitol, xylisorb, fine granular; the isomalt pf, palatinit; the carnauba wax; and the crystalac c 3.2. The dissolvable toothpaste bean-shaped system permits a user-consumer to evenly distribute toothpaste all around a mouth portion, teeth, and a gum region of the user-consumer while reducing the amount of the toothpaste that gets wasted and thrown away when stored in a tube. The toothpaste is located in a center and is semi-solid thereby permitting the user-consumer to chew while the dissolvable toothpaste bean-shaped system is dissolving in the mouth while the user is brushing teeth of the dissolvable toothpaste bean-shaped system.

The toothpaste comprises approximately 54.0967 percent weight and 336 grams total weight of the dissolvable toothpaste bean-shaped system. The gum arabic solution go-8-19018 comprises approximately 3.036499 percent weight and 18.86 grams total weight of the dissolvable toothpaste bean-shaped system. The titanium powder, argo 0-200 10-50-0d comprises approximately 0.181932 percent weight and 1.13 grams total weight of the dissolvable toothpaste bean-shaped system. The pepper mint flavor, triple distilled comprises approximately 0.322004 percent weight and 2 grams total weight of the dissolvable toothpaste bean-shaped system. The xylitol, xylisorb, fine granular comprises approximately 37.03048 percent weight and approximately 230 grams total weight of the dissolvable toothpaste bean-shaped system. The isomalt pf, palatinit comprises approximately 4.830062 percent weight and approximately 30 grams total weight of the dissolvable toothpaste bean-shaped system.

The carnauba wax comprises approximately 0.099821 percent weight and approximately 0.62 grams total weight of said dissolvable toothpaste bean-shaped system. The crystalac c 3.2 comprises approximately 2.5 percent weight and approximately 0.402505 grams total weight of said dissolvable toothpaste bean-shaped system. The dissolvable toothpaste bean-shaped system comprises a total weight of approximately 621.11 grams total weight per each dissolvable toothpaste bean-shaped system. The at least one confectioners glaze provides a semi-hard outer shell while providing the dissolvable toothpaste bean-shaped system with stability and a glossy shine. The at least one confectioners glaze comprises approximately 0.25 to 1 percent depending on size of the dissolvable toothpaste bean-shaped system. The dissolvable toothpaste bean-shaped system is sugar-free. The dissolvable toothpaste bean-shaped system is non-toxic if swallowed.

A method of producing a dissolvable toothpaste bean-shaped system is disclosed herein comprising the steps of: depositing toothpaste into a starch mold; placing the starch mold into a warm box at 110 degrees Fahrenheit until firm, approximately 3 days; mixing titanium powder into gum solution; placing centers into a revolving pan; applying gum solution to evenly coat the centers; evenly sprinkling and dry charging the centers with crystaline xylitol to dry centers (applying only enough of the crystaline xylitol to evenly coat); allowing the centers to tumble and sweat back; applying additional crystaline xylitol until all of the crystaline xylitol is used; allowing the dissolvable toothpaste bean-shaped system to sweat back further; applying the isomalt powder; drying the dissolvable toothpaste bean-shaped system overnight in the revolving pan; applying caranuba wax and allowing to tumble until a shine is developed; and applying confectioners glaze in liquid form using a ladle while the revolving pan is rotating.

The present invention holds significant improvements and serves as a dissolvable toothpaste bean-shaped system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, dissolvable toothpaste bean-shaped system (Tooth Bean System), constructed and operative according to the teachings of the present invention.

FIG. 1 shows a perspective view illustrating a dissolvable toothpaste bean-shaped system in an in-use condition according to an embodiment of the present invention.

FIG. 2 shows a perspective view illustrating a dissolvable toothpaste bean-shaped system in another in-use condition using a toothbrush according to an embodiment of the present invention.

FIG. 3 is a perspective view illustrating the dissolvable toothpaste bean-shaped system according to an embodiment of the present invention of FIGS. 1 & 2.

FIG. 4 is a flowchart illustrating a composition of the dissolvable toothpaste bean-shaped system according to an embodiment of the present invention.

FIG. 5 is a flowchart illustrating a method of producing the dissolvable toothpaste bean-shaped system according to an embodiment of the present invention of FIGS. 1-4.

FIG. 6 shows a side view illustrating a toothbrush to be used in conjunction with the dissolvable toothpaste bean-shaped system of the present invention.

The various embodiments of the present invention will hereinafter be described in conjunction with the appended drawings, wherein like designations denote like elements.

### DETAILED DESCRIPTION

As discussed above, embodiments of the present invention relate to an oral hygiene device and more particularly to a dissolvable toothpaste bean-shaped system (Tooth Bean System) to permit a user-consumer to evenly distribute toothpaste all around a mouth portion, teeth, and a gum region of the user-consumer while reducing the amount of toothpaste that gets wasted and thrown away when stored in a tube.

Generally speaking, small pieces of dentifrice (dissolvable toothpaste bean-shaped system) are preferably created using either using a traditional toothpaste or a modified dentifrice toothpaste. The final toothpaste product will preferably be comprised of consistency that will be pliable enough to be formed into shapes and will maintain their basic shape. Individual pieces can be made in a variety of way.

The present invention is designed to evenly distribute toothpaste all around a person's mouth, teeth, and gums while reducing the amount of toothpaste that gets wasted and thrown away in the tube. The Tooth Bean System eliminates the mess and hassle associated with trying to squeeze toothpaste onto a toothbrush and can be offered in a variety of colors and flavors, appealing to both children and adults. The Tooth Bean System enables people to switch between flavors, preventing them from becoming bored with the same tube.

Each individual piece of sugar and/or sugar-free coated dentifrice/toothpaste will be a single serving utilized for purpose of cleansing an individual consumer's teeth. There may be a variety of flavors, colors and shapes to provide consumers variety. In terms of sensory, the individual toothpaste pieces will provide the consumer the practicality and oral hygiene of a dentifrice/toothpaste with the fun, variety and attraction of candy (example: jelly bean) via the various shapes, flavors, and colors. Additionally, this invention will provide consumers a convenient delivery of the dentifrice/toothpaste by taking the guess work out of how much toothpaste to use/squeeze out, eliminate the wasted dentifrice/toothpaste left in the tube, reduce waste that results from using too much. This invention will also provide consumers individual servings with various shapes, flavors and colors will entice users (including children) to brush their teeth and establish good oral hygiene. The product also provides users with the ability to bring sufficient servings to satisfy their needs, a convenience to business traveler, who of often are required to dispose of their toothpaste when passing through airport security.

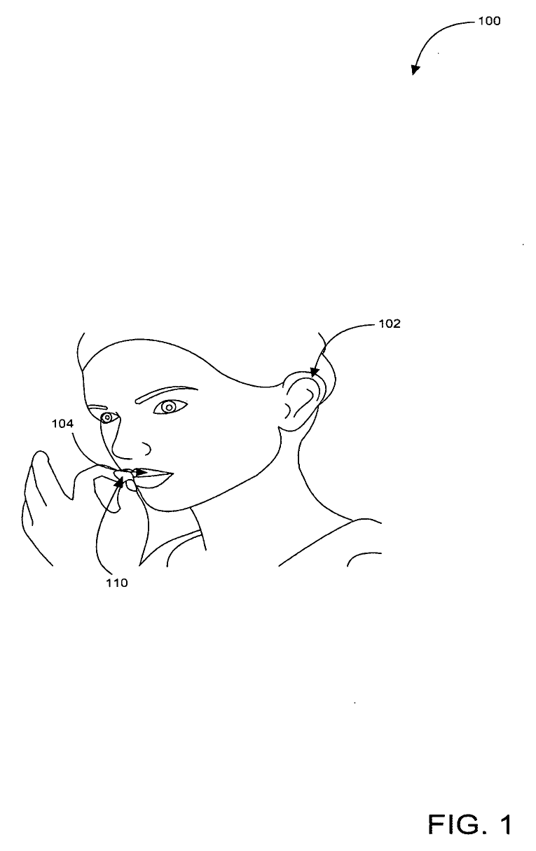

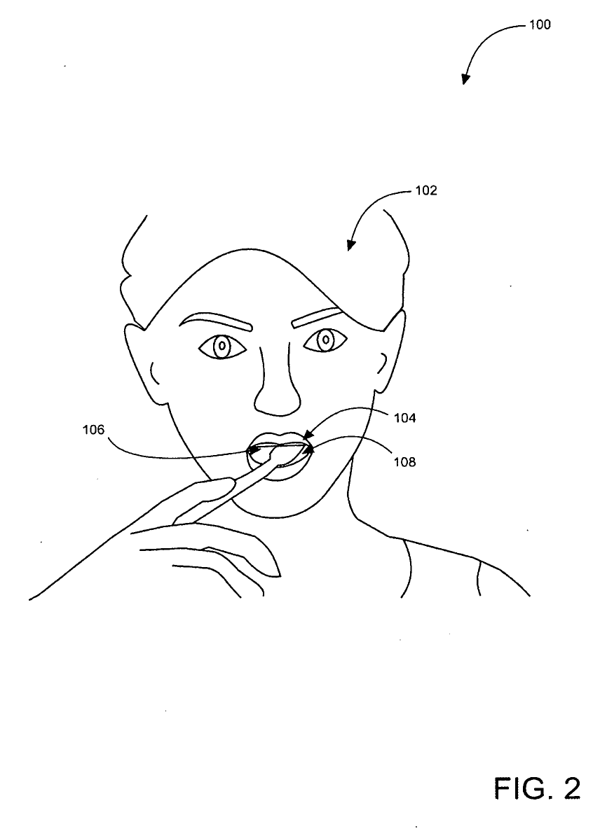

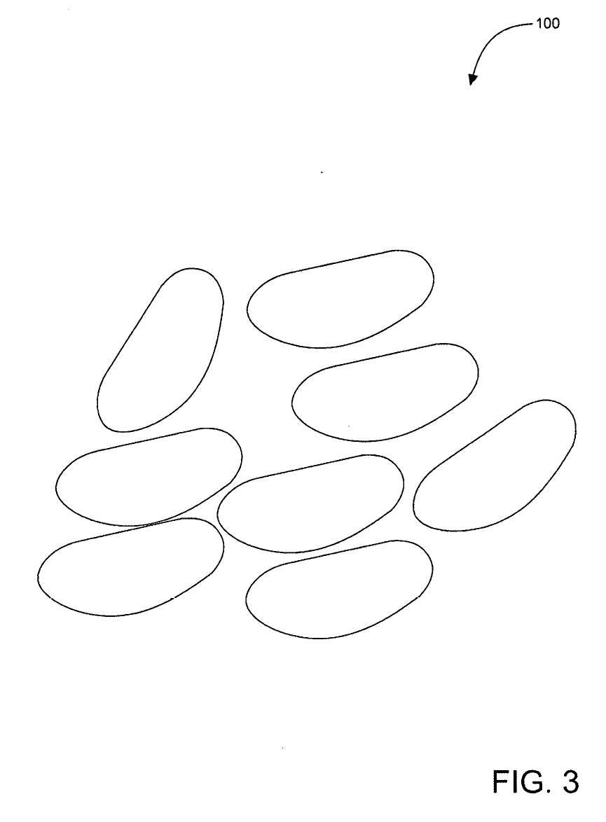

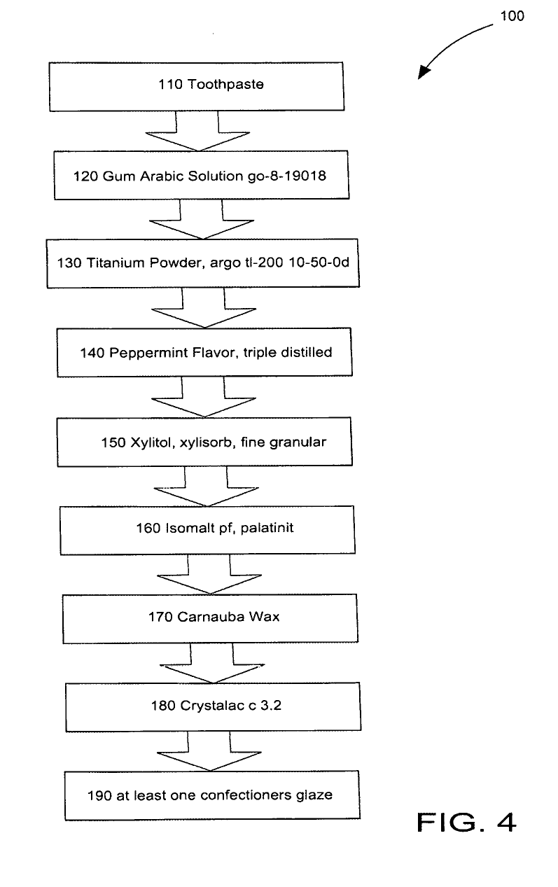

When referring to ‘bean-shaped’ the applicant is referring to curving and somewhat round in shape rather than jagged; “low rounded hills”; “rounded shoulders”, as shown in FIGS. 1-4 below.

Referring now to FIGS. 1-4 showing various perspective views of dissolvable toothpaste bean-shaped system 100 according to an embodiment of the present invention.

Dissolvable toothpaste bean-shaped system 100 comprises in a preferred embodiment ingredients combined in proportions substantially including: toothpaste 110; gum arabic solution go-8-19018 120; titanium powder, argo 0-200 10-50-0d 130; pepper mint flavor, triple distilled 140; xylitol, xylisorb, fine granular 150; isomalt pf, palatinit 160; carnauba wax 170; crystalac c 3.2 180; and at least one confectioners glaze 190. Dissolvable toothpaste bean-shaped system 100 is prepared by intermixing toothpaste 110; gum arabic solution go-8-19018 120; titanium powder, argo 0-200 10-50-0d 130; pepper mint flavor, triple distilled 140; xylitol, xylisorb, fine granular 150; isomalt pf, palatinit 160; carnauba wax 170; and crystalac c 3.2 180. Dissolvable toothpaste bean-shaped system 100 permits user-consumer 102 to evenly distribute toothpaste all around mouth portion 104, teeth 106, and gum region 108 of user-consumer 102 while reducing amount of toothpaste 110 that gets wasted and thrown away when stored in a tube. Toothpaste 110 is located in a center and is semi-solid thereby permitting user-consumer 102 to chew while dissolvable toothpaste bean-shaped system 100 is dissolving in mouth portion 104 while user-consumer 102 is brushing teeth 106 of dissolvable toothpaste bean-shaped system 100.

Toothpaste 110 preferably comprises approximately 54.0967 percent weight and 336 grams total weight of dissolvable toothpaste bean-shaped system 100. Gum arabic solution go-8-19018 120 preferably comprises approximately 3.036499 percent weight and 18.86 grams total weight of dissolvable toothpaste bean-shaped system 100. Titanium powder, argo 0-200 10-50-0d 130 preferably comprises approximately 0.181932 percent weight and 1.13 grams total weight of dissolvable toothpaste bean-shaped system 100. Pepper mint flavor, triple distilled 140 preferably comprises approximately 0.322004 percent weight and 2 grams total weight of dissolvable toothpaste bean-shaped system 100. Xylitol, xylisorb, fine granular 150 preferably comprises approximately 37.03048 percent weight and approximately 230 grams total weight of dissolvable toothpaste bean-shaped system 100. Isomalt pf, palatinit 160 comprises approximately 4.830062 percent weight and approximately 30 grams total weight of dissolvable toothpaste bean-shaped system 100.

Carnauba wax 170 comprises approximately 0.099821 percent weight and approximately 0.62 grams total weight of dissolvable toothpaste bean-shaped system 100. Crystalac c 3.2 180 preferably comprises approximately 2.5 percent weight and approximately 0.402505 grams total weight of dissolvable toothpaste bean-shaped system 100. Dissolvable toothpaste bean-shaped system 100 comprises a total weight of approximately 621.11 grams total weight per each dissolvable toothpaste bean-shaped system 100. At least one confectioners glaze 190 provides a semi-hard outer shell while providing dissolvable toothpaste bean-shaped system 100 with stability and a glossy shine. At least one confectioners glaze 190 preferably comprises approximately 0.25 to 1 percent depending on size of dissolvable toothpaste bean-shaped system 100. Dissolvable toothpaste bean-shaped system 100 may be sugar-free in certain embodiments. Further, dissolvable toothpaste bean-shaped system 100 is non-toxic if swallowed.

The toothpaste bean-shaped system 100 may be formed using ingestible or edible toothpaste. One suitable formula for such an edible toothpaste contains:

1200 grams of soluble sodium metaphosphate;

200 grams of dicatcium phosphate;

1160 grams of glycerin;

50 grams of sodium carboxynathylcellulose;

4 grams of saccharin;

1390 grams of water; and

16 grams of oil of spearmint.

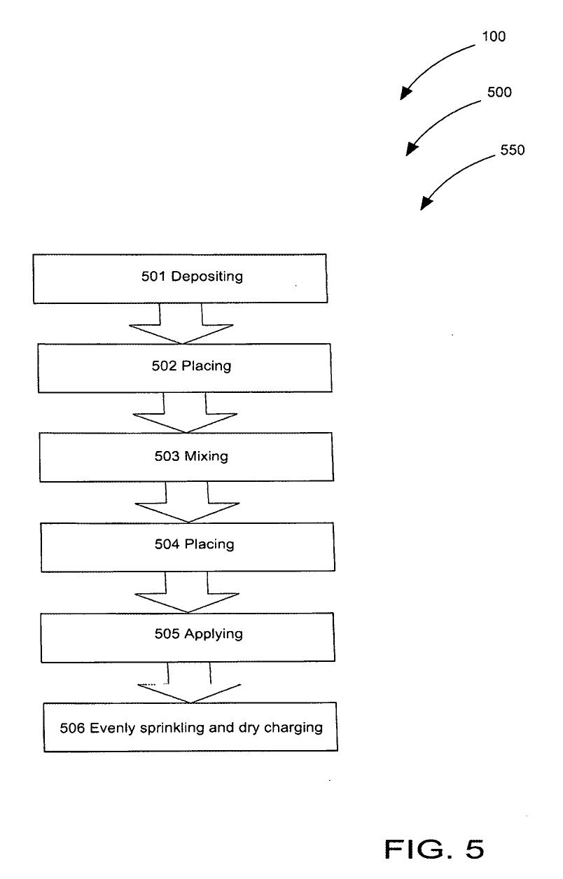

Referring now to FIG. 5, showing flowchart 550 illustrating method of producing 500 dissolvable toothpaste bean-shaped system 100 according to an embodiment of the present invention of FIGS. 1-4.

Method of producing 500 dissolvable toothpaste bean-shaped system 100 preferably comprises the steps of: step one 501 depositing toothpaste 110 into a starch mold; step two 502 placing the starch mold into a warm box at 110 degrees Fahrenheit until firm, approximately 3 days; step three 503 mixing titanium powder 130 into gum solution 120; step four 504 placing centers into a revolving pan; step five 505 applying gum solution 120 to evenly coat the centers; step six 506 evenly sprinkling and dry charging the centers with crystaline xylitol to dry centers (applying only enough of the crystaline xylitol to evenly coat); step seven 507 allowing the centers to tumble and sweat back; step eight 508 applying additional crystaline xylitol until all of the crystaline xylitol is used; step nine 509 allowing dissolvable toothpaste bean-shaped system 100 to sweat back further; step ten 510 applying isomalt powder 160; step eleven 511 drying dissolvable toothpaste bean-shaped system 100 overnight in the revolving pan; step twelve 512 applying caranuba wax 170 and allowing to tumble until a shine is developed; and step thirteen 513 applying confectioners glaze 190 in liquid form using a ladle while the revolving pan is rotating.

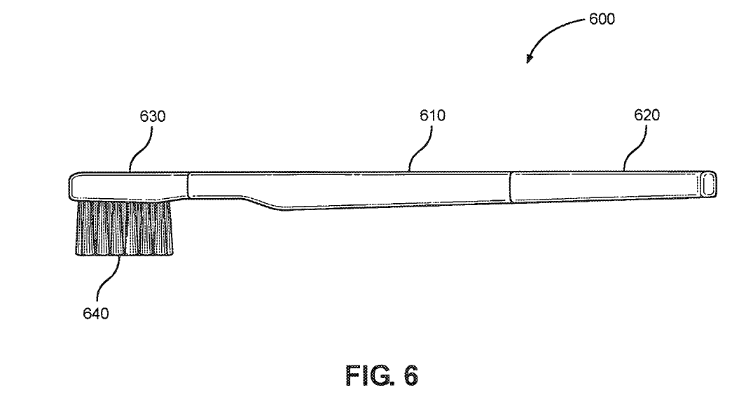

Referring now to FIG. 6, the instant bean-shaped system is used in conjunction with a toothbrush 600 to achieve improved oral hygiene with the ease of a hassle-free (or convenient) single serving. The toothbrush 600 comprises an elongated body 610 including a handle end 620 and a brush end 630 spaced from the handle end, and a plurality of bristle members 640, wherein the plurality of bristle members 640 are located on the brush end 630 of the elongated body 610 and are adapted to evenly distribute the dissolvable toothpaste all around a mouth portion, teeth, and a gum region of a user-consumer.

To further describe the chemical compounds used within the scope of the instant invention, the following information is provided and incorporated herein:

Chemical and Known Names for Xylisorb:

xylitol; ribitol; adonitol; Xylite; D-Xylitol; Adonite; Adonit; D-ribitol 488-81-3; Eutrit; Klinit; Xyliton; Xylite (sugar); Kannit, Newtol, 87-99-0; Pentitol, 1,2,3,4,5-pentanepentol; Xylit, Fluorette; Xylisorb; Kylit; meso-ribitol; meso-xylitol; xylo-Pentitol; L-ribitol; L-xylitol; Xylitol C; Xylitab DC; UNII-353ZQ9TVDA; UNII-VCQ006KQ1E; Wood sugar alcohol; Xylisorb 300; Xylisorb 700; Xylitab 100; Xylitab 300; CHEBI:15963; CHEBI:17151; (2R,4S)-pentane-1,2,3,4,5-pentol; (2R,3R,4S)-Pentane-1,2,3,4,5-pentaol; EINECS 201-788-0; NSC 25283; BRN 1720523; 353ZQ9TVDA; (2R,3s,4S)-pentane-1,2,3,4,5-pentol; (2R,3S,4S)-Pentane-1,2,3,4,5-pentaol; HEBKCHPVOIAQTA-ZXFHETKHSA-N; Ribito;, Adonite; 488-81-3; MFCD00064291; MFCD00064292; ST50411707; 4-01-00-02832 (Beilstein Handbook Reference); pentane-1,2,3,4,5-pentol; 1,2,3,4,5-Pentahydroxypentane; C5H12O5; EINECS 207-685-7; NSC 16868; BRN 1720524; WURCS=1.0/1,0/[h111h]; WURCS=1.0/1,0/[h121h]; 16277-71-7; Xylitol [USAN]; Adonitot; Adonitrot; Adonitt; Pentitot; D-Adonitol; RB0; Adonitol (7CI); AC1L1NKP; bmse000062; bmse000129; bmse000886; Epitope ID:114702; Epitope ID:114703; AC1Q59QK; SCHEMBL4250; DSSTox_CID_22514; DSSTox_RID_80046; VCQ006KQ1E; DSSTox_GSID_42514; SCHEMBL15318; 1,2,3,4,5-Pentanot; MLS002695898; 1,2,3,4,5-Pentanepentot; 47844_SUPELCO; A5502_SIGMA; CHEMBL96783; Ribitol (6CI,8CI,9CI); W507930_ALDRICH; X3375_SIGMA; A5502_SIAL; QSPL 191; SCHEMBL1924966; CHEMBL1865120; CHEMBL3137744; DTXSID7042514; 02240_FLUKA; HMDB00508; HMDB02917; HEBKCHPVOIAQTA-SCDXWVJYSA-N; MolPort-003-895-940; MolPort-003-934-322; 12426-00-5; HY-N0538; Tox21_201056; AR-117080; ZINC18068098; 1,2,3,4,5-PentahydroxypentaneXylite; AKOS015903403; AKOS015915193; ZINC100014205; Z1NC100018612; CCG-214167; CS-2987; CS-6043; DB01904; CAS-87-99-0; (2S,4R)pentane-1,2,3,4,5-pentaol; NCGC00165982-01; NCGC00165982-02; NCGC00258609-01; AK307317; AK307743; BT000167; CA000124; E967; K573; SMR001562099; (2S,4R)-pentane-1,2,3,4,5-pentol; HY-100582; LS-162656; A0171; FT-0631414; N1725; (2R,3r,4S)-pentane-1,2,3,4,5-pentol; (2R,3s,4S)-Pentane-1,2,3,4,5-pentot; 2088-EP2272822A1; 2088-EP2275417A2; 2088-EP2284162 A2; 2088-EP2284163A2; 2088-EP2292234A1; 2088-EP2292612A2; 2088-EP2301938 A1; 2088-EP2305254A1; 2088-EP2311842A2; 2088-EP2314584A1; 2088-EP2371811 A2; 2088-EP2377849A2; C00379; C00474; K-9195; X-7000; 127873-EP2275398 A1; 127873-EP2295402A2; 127873-EP2374783A1; 127873-EP2377841A1; 114-7373; 114-19011; 114-19012; UNII-YFV05Y57M9 component HEBKCHPVOIAQTA-SCDXWVJYSA-N; UNII-YFV05Y57M9 component HEBKCHPVOIAQTA-ZXFHETKHSA-N; 5DCF4F57-E023-469A-B4F3-91E8349A6705; 6684F574-C267-40CB-8828-12F2550E58D0; 28296-13-1; 37191-59-6; 7313-55-5; 75398-81-1; 84709-28-4; and 84709-42-2.

Chemical and Known names for Isomalt: Isomalt; 1-O-alpha-D-Glucopyranosyl-D-mannitol; Isomaltitol; Palatinitol; Palatinit; 20942-99-8; CCRIS 3698; 64519-82-0; Isomaltidex; GalenIQ 980; a-D-Glcp-1,1-D-Mannitol; 1-O-beta-D-glucopyranosyl-D-mannitol; HSDB 7969; 6-O-alpha-D-Glucopyranosyl-D-glucitol mixed with 1-O-alpha-D-glucopyranosyl-D-mannitol; AC1L3GDI; DSSTox_CID_753; DSSTox_RID_75770; DSSTox_GSID_20753; SCHEMBL154031; UNII-G97P6S66E9; 1-o-d-glucopyranosyl-d-mannitol; CHEMBL3187473; DTXSID60872321; D-Glucitol, 6-O-alpha-D-glucopyranosyl-, mixed with 1-O-alpha-D-glucopyranosyl-D-mannitol; D-Glucitol, 6-O-alpha-D-glucopyranosyl-, mixt. with 1-O-alpha-D-glucopyranosyl-D-mannitol; G97P6S66E9; 1-0-a-D-glucopyranosyl-D-mannitol; EINECS 244-122-4; Tox2113 200914; MFCD03414194; ZINC31319586; AKOS027379116; NCGC00248874-01; NCGC00258468-01; AK387289; AN-38770; CA001556; CA005360; CA005361; D-Mannitol, 1-O-alpha-D-glucopyranosyl-; CAS-64519-82-0; W-109182; 1-O-ALPHA-D-GLUCOPYRANOSYL-D-MANNITOL. DIHYDRATE; 570FC73A-80F5-464F-B47E-B9B4CC544121; UNII-3A0A6EFR5I component SERLAGPUMNYUCK-DCUALPFSSA-N; WURCS=1.0/2,1/[22122hl1,5][h1122h]1+1,2+1; (2R,3R,4R,5R)-6-(((2S,3R,4S,5S,6R)-3,4,5-Trihydroxy-6-(hydroxymethyl)tetrahydro-2H-pyran-2-yl)oxy)hexane-1,2,3,4,5-pentaol; (2R,3R,4R,5R)-6-[(2S,3R,4S,5S,6R)-3,4,5-trihydroxy-6-(hydroxymethyl)oxan-2-yl]oxyhexane-1,2,3,4,5-pentol; (2R,3R,4R,5R)-6-[(2S,3R,4S,5S,6R)-3,4,5-trihydroxy-6-(hydroxymethyl)tetrahydropyran-2-yl]oxyhexane-1,2,3,4,5-pentol; 124569-57-9; 124569-59-1; 177766-79-9; 746666-13-7; 852954-37-1; and 874794-40-8.

Chemical and Known Names for Crystalac c 3.2:

SHELLAC; Schellack; Lacca; Candy glaze; Lac resin; Gum lac; Shellac, purified; Confectioner's glaze; LS-2463; Shellac orange S-40; Resins, lac or shellac; and EINECS 232-549-9.

It should be noted that the steps described in the method of use can be carried out in many different orders according to user preference. The use of “step of” should not be interpreted as “step for”, in the claims herein and is not intended to invoke the provisions of 35 U.S.C. §112, ¶6. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A combination of a toothbrush and a dissolvable toothpaste bean-shaped system, said combination comprising:
a toothbrush including:
an elongated body;
wherein said elongated body includes a handle end and a brush end spaced from said handle end; and
a plurality of bristle members;
wherein said plurality of bristle members are located on said brush end of said elongated body and are adapted to evenly distribute a dissolvable toothpaste all around a mouth portion, teeth, and a gum region of a user-consumer; and
a dissolvable toothpaste bean-shaped system comprising:
toothpaste;
gum arabic solution;
titanium powder;
pepper mint flavor, triple distilled;
XYLITOL, XYLISORB, fine granular;
ISOMALT, PALATINIT;
CARNAUBA wax; and
CRYSTALAC;
wherein said dissolvable toothpaste bean-shaped system is prepared by intermixing said toothpaste; said gum arabic solution; said titanium powder; said pepper mint flavor, triple distilled; said XYLITOL, XYLISORB, fine granular; ISOMALT, PALATINIT; said CARNAUBA wax; and said CRYSTALAC;
wherein said dissolvable toothpaste bean-shaped system permits said user-consumer to evenly distribute toothpaste all around said mouth portion, teeth, and gum region of said user-consumer while reducing the amount of said toothpaste that gets wasted and thrown away when stored in a tube; and
wherein said dissolvable toothpaste bean-shaped system is used in combination with said toothbrush to evenly distribute toothpaste all around said mouth portion, teeth, and gum region of said user-consumer.

2. The combination of claim 1 wherein said toothpaste comprises approximately 54.0967 percent weight and 336 grams total weight of said dissolvable toothpaste bean-shaped system.

3. The combination of claim 1 wherein said toothpaste is located in a center and is semi-solid thereby permitting said user-consumer to chew while said dissolvable toothpaste bean-shaped system is dissolving in said mouth while said user is brushing teeth of said dissolvable toothpaste bean-shaped system.

4. The combination of claim 1 wherein said gum arabic solution comprises approximately 3.036499 percent weight and 18.86 grams total weight of said dissolvable toothpaste bean-shaped system.

5. The combination of claim 1 wherein said titanium powder, comprises approximately 0.181932 percent weight and 1.13 grams total weight of said dissolvable toothpaste bean-shaped system.

6. The combination of claim 1 wherein said pepper mint flavor, triple distilled comprises approximately 0.322004 percent weight and 2 grams total weight of said dissolvable toothpaste bean-shaped system.

7. The combination of claim 1 wherein said XYLITOL, XYLISORB, fine granular comprises approximately 37.03048 percent weight and approximately 230 grams total weight of said dissolvable toothpaste bean-shaped system.

8. The combination of claim 1 wherein said ISOMALT, PALATINIT comprises approximately 4.830062 percent weight and approximately 30 grams total weight of said dissolvable toothpaste bean-shaped system.

9. The combination of claim 1 wherein said CARNAUBA wax comprises approximately 0.099821 percent weight and approximately 0.62 grams total weight of said dissolvable toothpaste bean-shaped system.

10. The combination of claim 1 wherein said CRYSTALAC comprises approximately 2.5 percent weight and approximately 0.402505 grams total weight of said dissolvable toothpaste bean-shaped system.

11. The combination of claim 1 wherein said dissolvable toothpaste bean-shaped system comprises a total weight of approximately 621.11 grams total weight per each said dissolvable toothpaste bean-shaped system.

12. The combination of claim 1 wherein said dissolvable toothpaste bean-shaped system is sugar-free.

13. The combination of claim 1 wherein said dissolvable toothpaste bean-shaped system is non-toxic if swallowed.

14. The combination of claim 1 further comprises at least one confectioners glaze for providing a semi-hard outer shell.

15. (canceled)

16. (canceled)

17. (canceled)
